(19) United States
(12) Patent Application Publication
(10) Pub. No.: US 2007/0076842 A1
(43) Pub. Date: Apr. 5, 2007

(54) Adaptable energy discriminating computed tomography system

(76) Inventors: John Eric Tkaczyk, Delanson, NY (US); Deborah Joy Walter, Burnt Hills, NY (US); Yanfeng Du, Rexford, NY (US); David Michael Hoffman, New Berlin, WI (US); Thomas Louis Toth, Brookfield, WI (US)

Correspondence Address: Patrick S. Yoder;FLETCHER YODER, P.O. Box 692289, Houston, TX, 77269-2289, US

(21) Appl. No.: 11/239,842

(22) Filed: Sep. 30, 2005

(51) Int. Cl. H05G 1/60; H05G 1/44; A61B 6/00; G01N 23/00; G21K 1/12

(52) U.S. Cl. 378/5; 378/108

(57) ABSTRACT

A method of enhancing image quality and providing tissue composition information by analysis of energy discrimination data, the method comprising determining a radiation dosage at one or more energy spectrum levels based on patient parameters and user selected parameters. Computer-readable medium and systems that afford functionality of the type defined by this method are also contemplated in conjunction with the present technique.

### BACKGROUND

The invention relates generally to an energy discriminating computed tomography (CT) system, and more particularly to enhancing image quality in CT systems using detectors for high flux rate imaging with photon counting and energy discrimination.

Radiographic imaging systems, such as X-ray and computed tomography (CT) have been employed for observing, in real time, interior aspects of objects. Typically, the imaging systems include an X-ray source that is configured to emit X-rays toward an object of interest, such as a patient, a work piece, a parcel, a piece of luggage, and so forth. A detecting device, such as an array of radiation detectors, is positioned on the other side of the object and is configured to detect the X-rays transmitted through the object.

Patient absorbed X-ray dose is a major concern for CT imaging. Each CT acquisition protocol is constructed as a compromise in dose delivered to a patient versus the image quality measured as contrast to noise ratio (CNR). As will be appreciated, for a given detection equipment, acquisition protocol and image reconstruction and/or processing algorithm, a higher X-ray dose results in less noise and higher contrast CNR.

Conventional CT imaging systems utilize detectors that convert radiographic energy into electric charge signals that are integrated over a time period, then measured and ultimately digitized. A drawback of such detectors however is their inability to provide data or feedback as to the number and/or energy of photons detected. Also, energy discriminating, direct conversion detectors that are more dose efficient and capable of not only X-ray counting, but also providing a measurement of the energy level of each X-ray detected have been employed in CT systems. As known in the art, such energy discriminating systems will place X-rays into one or more energy bins. One type of processing of energy bin values called Optimal Energy Weighting (OEW) will enhance the contrast to noise relative to the conventional CT system which employs an energy integration process. Another type of processing of multiple bin data is called Material Discrimination and is configured to extract quantitative tissue composition information, if sufficient photon statistics exist, process data from multiple energy bins. In other words, the photon counting detectors result in better image quality and new kinds of tissue composition information than the conventional charge-integration systems.

However, a drawback of these direct conversion semiconductor detectors is that these types of detectors cannot count at the X-ray photon flux rates typically encountered with conventional CT systems. Further, the very high X-ray photon flux rate has been known to cause pile-up and polarization that ultimately leads to detector saturation. “Pile-up” is a phenomenon that occurs when a source flux at the detector is so bright that there is a non-negligible possibility that two or more X-ray photons deposit charge packets in a single pixel (“photon pile-up”), or in neighboring pixels (“pattern pile-up”), during one read-out cycle (i.e., one frame). In such a case these events are incorrectly recognized as one single event having the sum of their energies. If this happens sufficiently often, this will result in a hardening of the spectrum as piled-up soft events are shifted in the bin to higher energy bins. Such hardening will generally degrade the accuracy of the material decomposition algorithms based on the analysis of photon count values in corresponding energy bins. In addition, pile-up leads to a more or less pronounced depression of counts in a central part of a bright source, resulting in flux loss. Further, the very high X-ray photon flux rate has been known to cause pile-up and polarization that ultimately leads to detector saturation. In other words, these detectors typically saturate at relatively low X-ray flux level thresholds. Above these thresholds, the detector response is not predictable or has degraded dose utilization.

Further, current systems are not configured to adapt their acquisition and processing protocols based on patient shape and size and anatomy to be imaged, factors that may affect the image quality and may lead to saturation of elements of the detector in certain locations (e.g., outside the trajectory lines of thicker or more dense tissues). In addition, current concepts for energy discrimination systems do not provide a mechanism to optimize photon statistics in multiple bins. Source settings, source filter material and thickness and the detector energy threshold settings may be adjusted to provide appropriate bin statistics. Similarly, systems currently do not optimize the tube voltage based on the patient shape and size and anatomy to be scanned. Previously conceived solutions to achieve higher contrast to noise ratios were configured to simply increase the flux rate and correspondingly the dose to the patient. As a result, the systems were constructed to operate with a very high dynamic range of X-ray flux rate which disadvantageously results in higher cost of system components, such as the detectors and the tube, to achieve this high dynamic range. In addition, to meet the demand for higher CNR in one or more energy bins, tubes with higher power were developed to provide this increasingly higher flux rate. However, the resulting tube is physically large and heavy because of the heat generated in the creation of X-rays. Additionally, the tube cooling allowances limit the time over which the tube may be operated at its highest power which may result in reduced patient flow.

There is therefore a need for an energy discriminating CT system that enhances image quality and provides sufficient photon count statistics in one or more energy bins to ensure statistically significant tissue composition information while reducing radiation dose to the patient. In particular, there is a significant need for a design that advantageously combines information about patient size, shape and anatomy to be imaged to optimize the acquisition and reconstruction protocols to enhance image quality and simultaneously reduce patient absorbed dose. Additionally, there is a particular need for optimizing processing and visualization algorithms in order to achieve higher CNR. Such optimizations will advantageously serve to reduce dose to the patient while providing comparable CNR.

### BRIEF DESCRIPTION

Briefly, in accordance with aspects of the present technique, a method of enhancing image quality and providing tissue composition information by analysis of energy discrimination data is presented. The method includes determining a radiation dosage at one or more energy spectrum levels based on patient parameters and user selected parameters. Computer-readable medium and systems that afford functionality of the type defined by this method are also contemplated in conjunction with the present technique.

In accordance with another aspect of the present technique a system for enhancing image quality and providing tissue composition information by analysis of energy discrimination data is presented. The system is configured to determine a radiation dosage at one or more energy spectral levels based on patient parameters and user selected parameters, where the system is configured to acquire patient parameters, receive user selected parameters, select system acquisition settings based on the patient parameters and the user selected parameters, by determining the system acquisition settings from multi-dimensional look-up tables based on the patient parameters and the user selected parameters, and where the system acquisition settings include a desirable scan rate, a source filter material and thickness, a tube voltage, a current or combinations thereof, acquire image data from the patient based on the selected settings, and process the image data.

In accordance with further aspects of the present technique a system for enhancing image quality and providing tissue composition information by analysis of energy discrimination data is presented. The system is configured to determine a radiation dosage at one or more energy spectrum levels based on patient parameters and user selected parameters.

In accordance with further aspects of the present technique a radiographic imaging system is presented. The system includes a radiation source configured to emit a stream of radiation toward a patient to be scanned. Further, the system includes a control system configured to enhance image quality by determining a radiation dosage based on patient parameters and user selected parameters. Additionally, the system includes a detector assembly configured to detect the stream of radiation and to generate one or more signals responsive to the stream of radiation, where the detector assembly includes one or more detectors configured to absorb radiation. The system also includes a system controller configured to rotate the radiation source and the detector assembly and to acquire one or more sets of projection data from the one or more detectors via a data acquisition system. Also, the system includes a computer system operationally coupled to the radiation source and the detector assembly, where the computer system is configured to receive the one or more sets of projection data.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an exemplary imaging system in the form of a CT imaging system for use in producing processed images;

FIG. 2 is a block diagram of a physical implementation of the CT system of FIG. 1;

FIG. 3 is a flow chart illustrating an exemplary process of enhancing image quality and providing tissue composition information by analysis of energy discrimination data for use in the system depicted in FIG. 1, in accordance with aspects of the present technique; and

FIGS. 4-7 are graphical representations of exemplary simulation results for variations of optimal contrast to noise ratio with respect to tube voltage, in accordance with aspects of the present technique.

### DETAILED DESCRIPTION

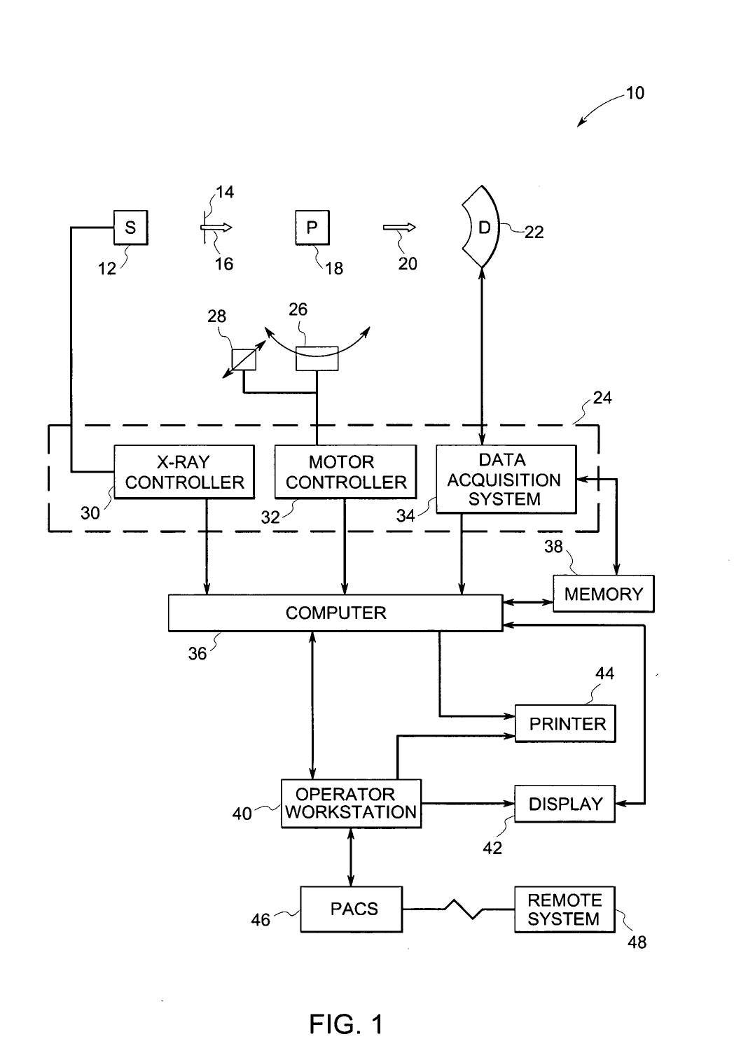

FIG. 1 is a block diagram showing an imaging system 10 for acquiring and processing image data in accordance with the present technique. In the illustrated embodiment, the system 10 is a computed tomography (CT) system designed to acquire X-ray projection data, to reconstruct the projection data into an image, and to process the image data for display and analysis in accordance with the present technique. In the embodiment illustrated in FIG. 1, the imaging system 10 includes a source of X-ray radiation 12. In one exemplary embodiment, the source of X-ray radiation 12 is an X-ray tube. The source of X-ray radiation 12 may include thermionic or solid-state electron emitters directed at an anode to generate X-rays or, indeed, any other emitter capable of generating X-rays having a spectrum and energy useful for imaging a desired object. Examples of suitable electron emitters include tungsten filament, tungsten plate, field emitter, thermal field emitter, dispenser cathode, thermionic cathode, photo-emitter, and ferroelectric cathode.

The source of radiation 12 may be positioned near a collimator 14, which may be configured to shape a stream of radiation 16 that is emitted by the source of radiation 12. The stream of radiation 16 passes into the imaging volume containing the subject to be imaged, such as a human patient 18. The stream of radiation 16 may be generally fan-shaped or cone-shaped, depending on the configuration of the detector array, discussed below, as well as the desired method of data acquisition. A portion 20 of radiation passes through or around the subject and impacts a detector array, represented generally at reference numeral 22. Detector elements of the array produce electrical signals that represent the intensity of the incident X-ray beam. These signals are acquired and processed to reconstruct an image of the features within the subject.

The radiation source 12 is controlled by a system controller 24, which furnishes both power, and control signals for CT examination sequences. Moreover, the detector 22 is coupled to the system controller 24, which commands acquisition of the signals generated in the detector 22. The system controller 24 may also execute various signal processing and filtration functions, such as for initial adjustment of dynamic ranges, interleaving of digital image data, and so forth. In general, system controller 24 commands operation of the imaging system to execute examination protocols and to process acquired data. In the present context, system controller 24 also includes signal processing circuitry, typically based upon a general purpose or application-specific digital computer, associated memory circuitry for storing programs and routines executed by the computer, as well as configuration parameters and image data, interface circuits, and so forth.

In the embodiment illustrated in FIG. 1, the system controller 24 is coupled via a motor controller 32 to a rotational subsystem 26 and a linear positioning subsystem 28. In one embodiment, the rotational subsystem 26 enables the X-ray source 12, the collimator 14 and the detector 22 to be rotated one or multiple turns around the patient 18. In other embodiments, the rotational subsystem 26 may rotate only one of the source 12 or the detector 22 or may differentially activate various stationary electron emitters to generate X-ray radiation and/or detector elements arranged in a ring about the imaging volume. In embodiments in which the source 12 and/or detector 22 are rotated, the rotational subsystem 26 may include a gantry. Thus, the system controller 24 may be utilized to operate the gantry. The linear positioning subsystem 28 enables the patient 18, or more specifically a patient table, to be displaced linearly. Thus, the patient table may be linearly moved within the gantry to generate images of particular areas of the patient 18.

Additionally, as will be appreciated by those skilled in the art, the source of radiation 12 may be controlled by an X-ray controller 30 disposed within the system controller 24. Particularly, the X-ray controller 30 is configured to provide power and timing signals to the X-ray source 12.

Further, the system controller 24 is also illustrated as including a data acquisition system 34. In this exemplary embodiment, the detector 22 is coupled to the system controller 24, and more particularly to the data acquisition system 34. The data acquisition system 34 receives data collected by readout electronics of the detector 22. The data acquisition system 34 typically receives sampled analog signals from the detector 22 and converts the data to digital signals for subsequent processing by a computer 36.

The computer 36 typically is coupled to or incorporates the system controller 24. The data collected by the data acquisition system 34 may be transmitted to the computer 36 for subsequent processing and reconstruction. The computer 36 may include or communicate with a memory 38 that may store data processed by the computer 36 or data to be processed by the computer 36. It should be understood that any type of memory configured to store a large amount of data might be utilized by such an exemplary system 10. Moreover, the memory 38 may be located at the acquisition system or may include remote components, such as network accessible memory media, for storing data, processing parameters, and/or routines for implementing the techniques described below.

The computer 36 may also be adapted to control features such as scanning operations and data acquisition that may be enabled by the system controller 24. Furthermore, the computer 36 may be configured to receive commands and scanning parameters from an operator via an operator workstation 40, which is typically equipped with a keyboard and other input devices (not shown). An operator may thereby control the system 10 via the input devices. Thus, the operator may observe the reconstructed image and other data relevant to the system from computer 36, initiate imaging, and so forth.

A display 42 coupled to the operator workstation 40 may be utilized to observe the reconstructed images. Additionally, the scanned image may also be printed by a printer 44, which may be coupled to the operator workstation 40. The display 42 and printer 44 may also be connected to the computer 36, either directly or via the operator workstation 40. The operator workstation 40 may also be coupled to a picture archiving and communications system (PACS) 46. It should be noted that PACS 46 might be coupled to a remote system 48, such as radiology department information system (RIS), hospital information system (HIS) or to an internal or external network, so that others at different locations may gain access to the image data.

It should be further noted that the computer 36 and operator workstation 40 may be coupled to other output devices, which may include standard or special purpose computer monitors and associated processing circuitry. One or more operator workstations 40 may be further linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, a virtual private network or the like.

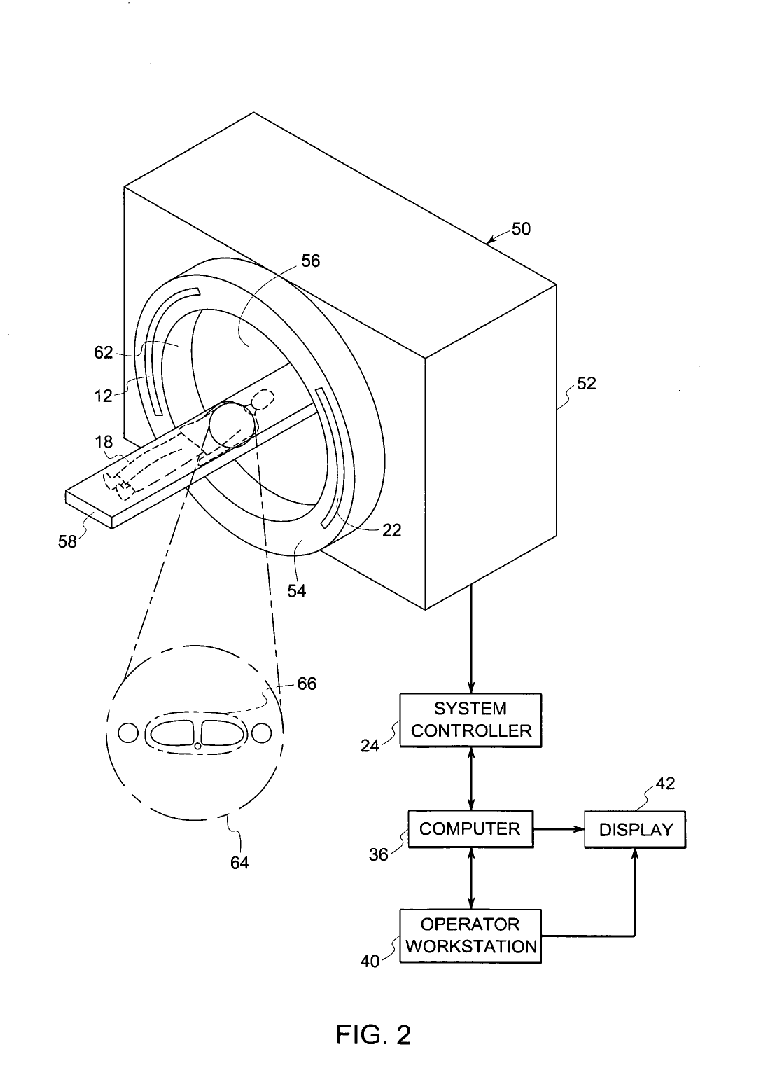

As noted above, an exemplary imaging system utilized in a present embodiment may be a CT scanning system 50, as depicted in greater detail in FIG. 2. The CT scanning system 50 may be a multi-slice CT (MSCT) system that offers a wide array of axial coverage, high rotational speed of the gantry, and high spatial resolution. Alternately, the CT scanning system 50 may be a volumetric CT (VCT) system utilizing a cone-beam geometry and an area detector to allow the imaging of a volume, such as an entire internal organ of a subject, at high or low gantry rotational speeds. The CT scanning system 50 is illustrated with a frame 52 and a gantry 54 that has an aperture 56 through which a patient 18 may be moved. A patient table 58 may be positioned in the aperture 56 of the frame 52 and the gantry 54 to facilitate movement of the patient 18, typically via linear displacement of the table 58 by the linear positioning subsystem 28 (see FIG. 1). The gantry 54 is illustrated with the source of radiation 12, such as an X-ray tube that emits X-ray radiation from a focal point 62. For cardiac imaging, the stream of radiation is directed towards a cross section of the patient 18 including the heart.

In typical operation, the X-ray source 12 projects an X-ray beam from the focal point 62 and toward detector array 22. The collimator 14 (see FIG. 1), such as lead or tungsten shutters, typically defines the size and shape of the X-ray beam that emerges from the X-ray source 12. The detector 22 is generally formed by a plurality of detector elements, which detect the X-rays that pass through and around a subject of interest, such as the heart or chest. Each detector element produces an electrical signal that represents the intensity of the X-ray beam at the position of the element during the time the beam strikes the detector. The gantry 54 is rotated around the subject of interest so that a plurality of radiographic views may be collected by the computer 36.

Thus, as the X-ray source 12 and the detector 22 rotate, the detector 22 collects data related to the attenuated X-ray beams. Data collected from the detector 22 then undergoes pre-processing and calibration to condition the data to represent the line integrals of the attenuation coefficients of the scanned objects. The processed data, commonly called projections, may then be filtered and backprojected to formulate an image of the scanned area. A formulated image may incorporate, in certain modes, projection data for less or more than 360 degrees of rotation of the gantry 54.

Once reconstructed, the image produced by the system of FIGS. 1 and 2 reveals internal features 66 of the patient 18. In traditional approaches for the diagnosis of disease states, and more generally of medical conditions or events, a radiologist or physician would consider the reconstructed image 64 to discern characteristic features of interest. In cardiac imaging, such features 66 include coronary arteries or stenotic lesions of interest, and other features, which would be discernable in the image, based upon the skill and knowledge of the individual practitioner. Other analyses may be based upon capabilities of various algorithms, including algorithms generally referred to as computer-aided detection or computer-aided diagnosis (CAD) algorithms.

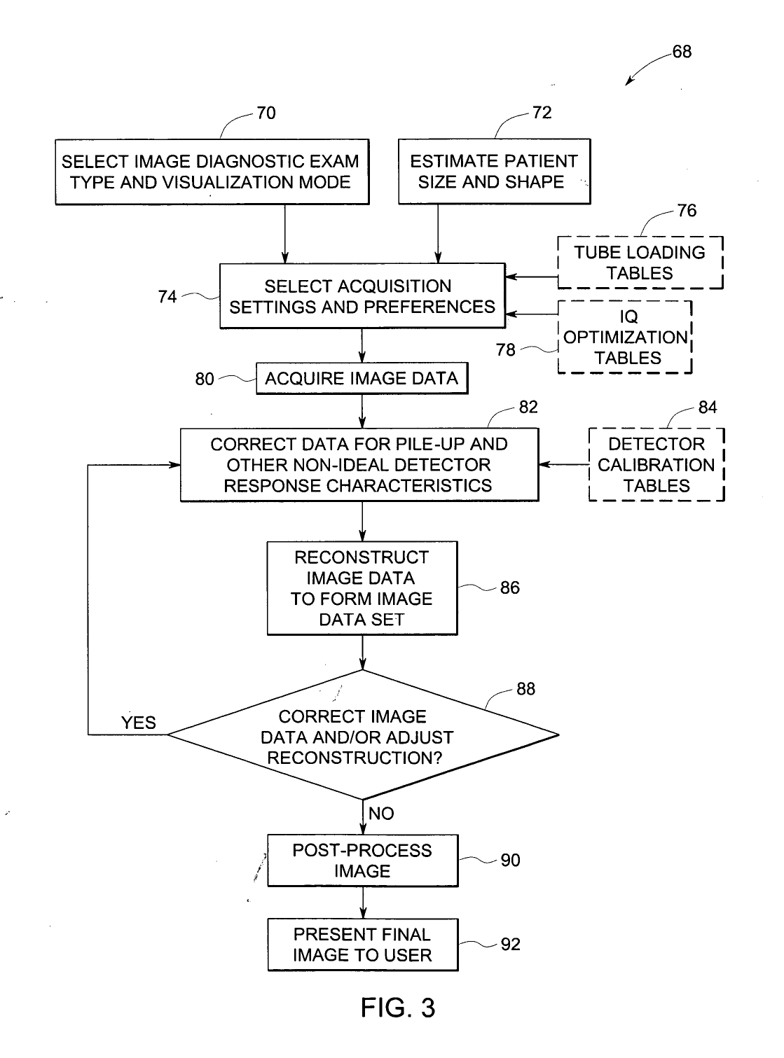

FIG. 3 is a flow chart of exemplary logic 68 for enhancing image quality for use in a system, such as system 10 depicted in FIG. 1. In accordance with exemplary aspects of the present technique, the method includes determining a radiation dosage based on patient parameters and user selected parameters. Additionally, the method utilizes information about patient size, shape and anatomical regions to be imaged to optimize acquisition protocol and reconstruction, processing and visualization algorithms to achieve a higher CNR² per unit dose. The method starts at step 70 where a user, such as a clinician, may select parameters representative of type of image diagnostic exam and mode of visualization, for example. These parameters may be referred to as user selected parameters.

For example, the type of image diagnostic exam may be based on a noise index. The noise index may include a low noise index or a high noise index, for example, when data for energy discrimination is acquired with two energy bins. A “noise index” may be generally defined as the expected standard deviation in image pixel values for soft tissue areas of the image. Such an index may be calculated based on measurements or simulations of a uniform water phantom with similar size and shape of patient but with water density adjusted to match the mean image value (i.e., CT number) of the actual data.

In addition, the visualization mode may correspond to a method of envisaging image data. The user may choose to visualize the image data as a two-dimensional image or a three-dimensional volumetric image, for example. Such visualization selections may influence the acquisition and/or processing of image data by modifying the way tissue contrast and noise in the acquired data is ultimately presented on the screen. More or less patient dose will be required to achieve an acceptable display of anatomic structure for any particular visualization mode requested by the system user.

In addition, the acquisition mode may be determined by the type of tissue the user may choose to discriminate. The user may choose to optimize the acquisition and post-processing algorithms for low-contrast enhancement of soft-tissue as in non-contrast enhanced examinations. Or the user may choose to optimize the acquisition and post-processing algorithms for iodine-enhanced contrast regions or bone imaging. By modifying the acquisition parameters more or less patient dose will be required to achieve acceptable CNR for the particular imaging task.

Further, with an energy discrimination CT system, acquired image data may be processed and displayed in a variety of ways which are different from conventional CT because of the additional tissue composition information provided by analysis of the energy discrimination data for material discrimination. Such ways may include presentation of material basis-set images as a pair of gray-scale images. Alternatively, in certain embodiments, a method of displaying acquired image data may include color-coded augmentation of conventional CT images to overlay tissue composition information on top of Hounsfield unit information. Depending upon the selected processing and display, again, the acquisition and/or processing of the data may be altered to ensure the best visualization of anatomic structure in the chosen mode. Typically, tissue composition information extracted by the analysis of energy discrimination data may require a data acquisition protocol and analysis which is configured to preserve good quantum statistics in each of the multi-energy bins. In particular, for systems with a direct conversion detector, it may be advantageous to use a source filter which effectively splits the X-ray energy spectrum into two lobes, one corresponding to the low energy bin and one with the high energy bin. Such a filter is composed of an element or elements with photoelectric k-edges which define the split energy. Such a filter may be used in the event that the material decomposition information is requested by the user. However, as will be appreciated, such a filter typically would not be utilized to optimize the CNR in conventional CT display mode.

Further, at step 72, parameters corresponding to estimates of patient size and shape may be acquired. In addition, information regarding an anatomical region of the patient being imaged may also be obtained. The patient size and shape and information regarding the anatomical region being imaged may be referred to as patient parameters. In one embodiment, estimates of the patient size and shape may be obtained from user input. However, in certain other embodiments, estimates of the patient size and shape may also be measured employing a visual imaging system, for example. On the other hand, estimates of the patient size and shape may be assessed from two orthogonal X-ray scout views obtained before the acquisition of image data. However, it should be noted that other methods of obtaining the estimates of the patient size and shape, such as, but not limited to, more than two-scout views at multiple angles, one high pitch helical scout scan, low resolution CT scans, acoustic range-finding and attenuation measurement instruments, weight-measurement or pressure sensors embedded into the patient table may also be employed. The patient parameters may be used to alter the acquisition and/or processing of the image data by, for example, selection of an optimal source kVp, generally using higher kVp as the patient size increases in order to ensure sufficient photon flux to the detector within the constraints of the source loading power limitation. The system may implement a source current (mA) and source filter modulation protocol in order to best match the patient shape and the associated projected flux profile onto the detector for each view of the CT sinogram. The source filter and detector energy bin settings may be adjusted so as to optimally account for the spectrum-hardening effects of the thicker patient size and achieve optimal split of photons into multiple energy bins to allow sufficient statistics to perform accurate material composition calculations.

Subsequently, at step 74, acquisition settings and preferences may be selected. Accordingly, preferred or optimal values of system acquisition settings and preferences may be selected based on the patient parameters and the user selected parameters. The system acquisition settings and preferences may include acquisition protocol, reconstruction algorithms, processing algorithms and visualization algorithms. In addition, the acquisition protocol may include a desirable scan rate, a source filter material and thickness, a tube voltage, a current or combinations thereof.

It is presently contemplated that a range of different system settings, acquisition protocols and reconstruction, processing, and visualization algorithms may be preselected based upon combinations of the various user selected and patient parameters. For example, for examination of the abdominal organs, the source kVp and mA may be adjusted to automatically to achieve a selected level of CNR in the image whereby a thick patient would require high kVp and high mA. In contrast, low dose arterial head exams involving iodine contrast would be implemented with a low kVp protocol, providing limited mA but yielding sufficient CNR to detail the arterial structures. The low kVp in this case may be the optimal dose strategy for iodine visualization due to the relatively low attenuation of the head and relatively high atomic number of iodine compared to soft body tissue. Optimal energy weighting (OEW) of the different energy bins may be used to enhance further the contrast to noise level for the iodine signal. Alternately, compositional studies of plaques in coronary vessels within a patient of large size may be performed with a higher dose protocol using a spectrum splitting, source filter and processing of two energy bin data necessary for compositional analysis. The system rotation may be slowed in order to accommodate the necessary statistics without over-ranging the photon counting detector. As explained above, compositional information may be overlaid on structure information as determined by the user selected color mapping algorithm.

Accordingly, using a combination of the patient parameters and the user selected parameters, optimal values of the system acquisition settings and preferences may be selected. As used herein, “optimal” values of the system acquisition settings and preferences are representative of the values of the system acquisition settings and preferences that are tailored based on the patient parameters and the user selected parameters, where the system acquisition settings are chosen to achieve higher CNR² per unit dose. Alternatively, such settings allow presentation of tissue compositional information. It should be noted that the system acquisition settings relations and preferences may be, and generally are selected prior to acquisition of image data via an imaging system, such as a CT system. It is presently contemplated that such settings and preferences will be stored, such as in look-up tables (see below) for later use in data acquisition.

The selecting step 74 may further include determining preferred or optimal system acquisition settings and preferences from such multi-dimensional look-up tables based on the patient parameters and the user selected parameters. In certain embodiments, the look-up tables may include tube loading tables 76 and/or image quality (IQ) optimization tables 78. The image quality tables 78 translate the user minimum requirements for soft tissue contrast and noise relative to that for bone or contrast agent into preferred ranges of acquisition parameters which are then modified by the allowed tube loading settings. Furthermore the need for quantitative tissue composition information will implement a multi-bin, energy discrimination protocol with spectrum splitting, source filter and material discrimination analysis. The tube loading tables 76 may include data representative of the power capability of the radiation source, such as the radiation source 12 (see FIG. 1). The tube loading tables 76 list the capability of the source to provide the requested mA at some kVp for a time required to complete the full CT scan over the anatomical region requested. Further, the tube loading tables 76 may be configured to provide information regarding settings of a tube (not shown) of the radiation source 12 based on the user selected parameters and the patient parameters. For example, the tube loading tables 76 may be configured to select a relatively higher tube voltage if the size of the patient is comparatively large. Alternatively, a lower tube voltage may be selected if the size of the patient is relatively small. Additionally, the IQ optimization tables 78 may include pre-computed information based on combinations of the user selected parameters, the patient parameters and information from the tube loading tables 76.

As previously noted, the system acquisition settings and preferences may include a scan rate of the system 10. In one embodiment, an optimal or preferred scan rate based on a combination of the user selected parameters, patient parameters and information from the tube loading tables 76 may be selected. Moreover, flux incident on the detectors 22 (see FIG. 1) for each view may be estimated prior to acquisition of image data. This estimation of incident flux facilitates selection of the preferred or optimal system acquisition settings and preferences. Consequently, the use of filters, both in hardware and software, such as a bowtie filter, a current profile and a scan rotation time may be selected so as to prevent exposure of the detectors 22 to incident flux beyond an associated saturation limit. A filter material may be implemented to appropriately split the spectrum and populate the energy bins so as to create optimal statistics for material decomposition algorithms. Such material decomposition algorithms generally need high photon statistics to generate accurate compositional information. Accordingly, an acquisition protocol may be customized such that the scan rate may be controlled automatically. For example, due to tube power limitations, it may be desirable to slow down the scan rate to facilitate collection of a greater number of photons by the detectors 22. Alternatively, the scan rate may be enhanced to avoid saturation of the detectors 22. In particular, if high number of counts is desirable to achieve a relatively good CNR, the system 10 (see FIG. 1) may be configured to rotate at a reduced rate so as to maintain the flux rate below a predetermined level. As the scan rate is relatively low, the scan is relatively long thereby ensuring that the desirable number of photons is delivered to the detectors 22 to achieve the desirable CNR in one or more energy bins. In accordance with exemplary aspects of the present technique, the scan rate may also be continuously varied during the scan in addition to scan to scan, or slice to slice to further optimize the detected count rates. Accordingly, the optimal scan rate may be configured to balance a high scan rate with a flux rate.

Following the selection of preferred or optimal system acquisition settings and preferences at step 74, image data may be acquired from the patient at step 80. The image data may be acquired from the patient in accordance with the selected system acquisition settings and preferences at step 80.

Subsequently, the acquired image data may be processed. Steps 82-92 depict an exemplary method of processing the acquired image data in accordance with exemplary aspects of the present technique. At step 82, the image data may be corrected for pile-up effects and/or other non-ideal detector response characteristics. As will be appreciated by those skilled in the art, direct conversion semiconductor detectors typically cannot count at the X-ray photon flux rates typically encountered with conventional CT systems. As previously noted “pile-up” is a phenomenon that occurs when a source flux at the detector is so bright that there is a non-negligible possibility that two or more X-ray photons deposit charge packets in a single pixel (“photon pile-up”), or in neighboring pixels (“pattern pile-up”), during one read-out cycle (i.e., one frame). Additionally, the very high X-ray photon flux rate has been known to cause pile-up and polarization that ultimately leads to detector saturation. In other words, these detectors typically saturate at relatively low X-ray flux level thresholds. Above these thresholds, the detector response is not predictable or has degraded dose utilization.

In one embodiment, a detector element may be pixelated into two separate sub-pixels that have different flux rate characteristics, for example. It should be noted that the terms detector element and pixel may be used interchangeably. In a conventional sense, then, the pixel generally represents the smallest area unit that may be resolved by the detector. In the present context, however, each “pixel” may be further broken down into sub-regions to improve the ability to count X-ray or high energy photons and thereby improve performance of the detector and avoid the effects of pile-up.

Furthermore, in one embodiment, the pixel may be pixelated into two separate sub-pixels that have different flux rate characteristics, for example. The pixel may include a first region having a first area and a second region having a second area. In one embodiment, the first area associated with the first region of the pixel may be substantially larger than the second area associated with the second region of the pixel. Consequently, the asymmetry in the areas associated with the sub-pixels results in a composite pixel area with different saturation thresholds within the composite pixel area.

It should also be noted that each of the first and second regions of the pixel may be configured to saturate at a predetermined level. Furthermore, each of the first and second regions of the pixel may also be configured to count photons received and associate an energy bin to each photon counted.

In accordance with exemplary aspects of the present technique, image data from each of the first and second regions may be acquired. Subsequently, the image data acquired via each of the first and second regions may be calibrated. In one embodiment, the image data acquired from each of the first and second regions may be calibrated by measuring the detected charge as a function of the input X-ray flux and fitting that function to a polynomial function. Pile-up effects and other non-ideal detector response characteristics may be corrected by applying a polynomial fit to the image data acquired from each of the first and second regions of the pixel, in one embodiment. Consequent to the application of this polynomial fit, the image data acquired from each of the first and second regions of the pixel may be corrected for pile-up effects. Look-up tables such as detector calibration tables 84 may be employed in facilitating the calibration and correction of pile-up effects and other non-ideal detector response characteristics. Furthermore, consequent to the calibration and correction steps the effective count rate capability of the pixel is enhanced albeit with a penalty of increased noise levels.

Subsequently, image data acquired from the first region of the pixel may be combined with the image data acquired from the neighboring, smaller-sized second region. The corrected image data from each of the first and second regions of the pixel may be weighted by an associated uncertainty. The uncertainty associated with the image data acquired from the first and second regions is a function of the number of detected photons and the detector quantum efficiency (DQE) of the detector. In one embodiment, the uncertainty may include noise associated with image data acquired from each of the first and second regions of the pixel. Following the weighting of the image data, the weighted image data associated with each of the first and second regions of the pixel may then be combined to generate composite image data.

In accordance with exemplary aspects of the present technique, at high count rates above which the first region of the pixel is saturated, only the image data from the second region of the pixel weighted by its associated uncertainty is assigned to a projection for the pixel. Alternatively, at low count rates, composite image data C representative of a weighted sum of the response from both the first and second regions is assigned to the projection for the pixel.

Additionally, at step 82, in the case of energy discriminating detectors, optimal energy weighting (OEW) and material decomposition (MD) sinograms may also be calculated employing calibration data. In other words, material data may be obtained from the energy associated with the image data. However, in the case of integrating detectors, beam hardening and detector specific spectral correction may be employed.

Following step 82, the image data may be reconstructed to form an image data set at step 86. Subsequently, at step 88, a check is carried out to verify whether further correction of the reconstructed image data is required. Additionally, at step 88 a check is also carried out to verify whether adjustment of the reconstruction of the image data is required. If correction and/or adjustment of the reconstructed image data are not entailed, the image reconstructed at step 86 may be post-processed at step 90. The post-processing step 90 may include a three-dimensional reformatting of the image. In addition, conventional and energy discriminating CT information may be combined into one image by overlaying the material information on the conventional image. In addition, image space-based filtering of the image may reduce image noise at the expense of spatial resolution. Because compositional information is available, image space-based filtering may be applied more heavily to the certain compositions, in particular, ones which otherwise due to the acquisition protocol are more noisy thereby optimizing the noise uniformity. For example, a low kVp technique to optimize iodine contrast tends to render soft tissue areas substantially noisy unless the soft tissue area is substantially heavily filtered in this post-processing step 90. It should also be noted that the visualization preferences selected by the user at step 70 may influence the acquisition and processing of the image data. A final image may then be presented to the user at step 92 in accordance with the visualization preferences selected by the user at step 70. With returning reference to the decision block 88, if correction of reconstructed image data and/or adjustment of reconstruction are required, steps 82-92 may be repeated thereby iteratively reconstructing the image data until a desirable image quality is achieved.

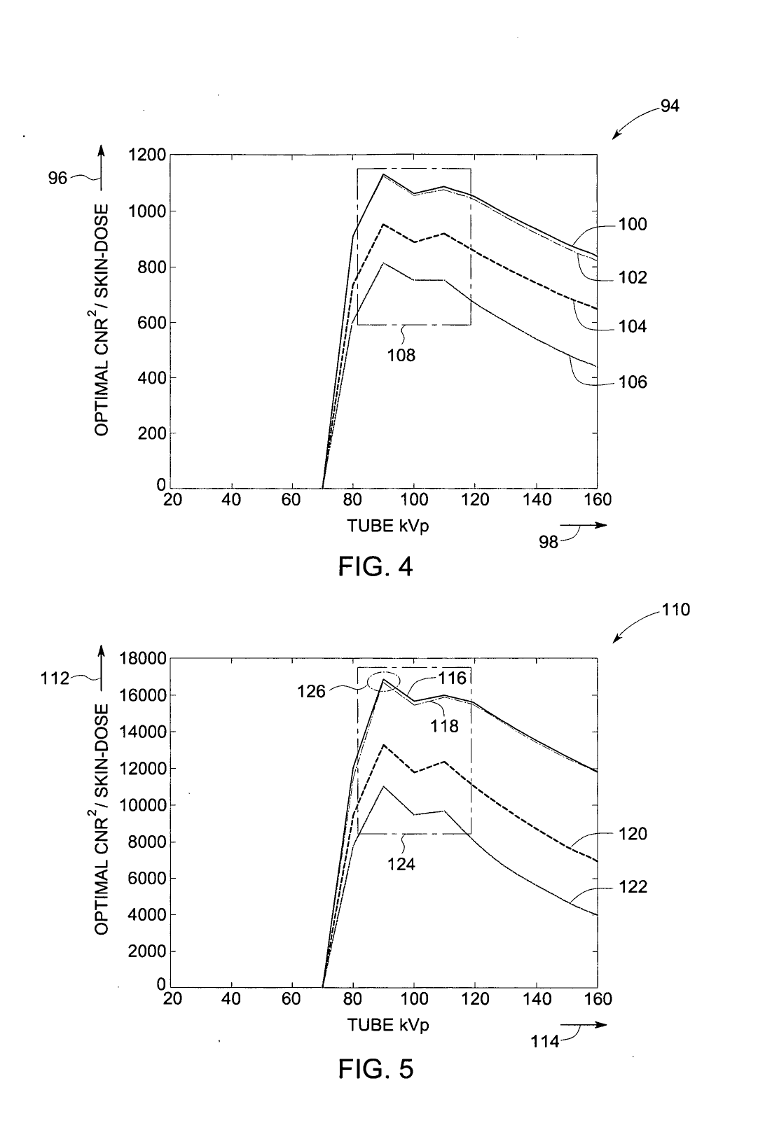

FIGS. 4-7 are graphical representations of simulation results where optimal tube voltage settings are obtained for different target tissues to be imaged. Such representations may be incorporated into the IQ Optimization tables 78 (see FIG. 3). In FIG. 4, a graphical representation of simulation results 94 depicting a variation in an optimal CNR² per skin-dose 96 is plotted against a variation in tube voltage 98. Also, in FIG. 4, the target tissue or the anatomy being imaged may include bones and for a patient size about 30 cm. Response curve 100 represents a variation of the optimal CNR² per skin-dose 96 as a function of the tube voltage 98 for the case where energy bin data is processed by an optimal energy weighting (OEW) algorithm, where the weight w is equal to the difference of X-ray attenuation (Δμ) between bone μ_{bone} and water μ_{water}. In other words w=Δμ=μ_{bone}-μ_{water}).

Response curve 102 embodies a variation of the optimal CNR² per skin-dose 96 as function of the tube voltage 98 for another OEW algorithm, where the weight w is inversely scaled with the cube of energy (E), w=1/E³. Additionally, response curve 104 corresponds to a variation of the optimal CNR² per skin-dose 96 as a function of the tube voltage 98 for a photon counting system, where the weight is the same for each energy bin, in other words, w=1. Also, response curve 106 signifies a variation of optimal CNR² per skin-dose 96 as a function of the tube voltage 98 for a conventional energy integrating system, where the bin weight is proportional to energy, in other words, w=E. Furthermore, reference numeral 108 embodies an optimal region of operating the tube. In the illustrated embodiment depicted in FIG. 4, an optimal tube voltage for imaging bones for a patient size of about 30 cm may be in a range from about 80 kVp to about 120 kVp. Furthermore, the higher CNR² per skin-dose 96 corresponding to the OEW curves 100 and 102 indicate that this processing of the energy bin data may be optimal. Moreover, in accordance with aspects of the present technique, this information regarding the optimal tube voltage settings may be added to the IQ optimization tables 78 (see FIG. 3).

FIG. 5 illustrates a graphical representation of simulation results 110 where a variation in an optimal CNR² per skin-dose 112 is plotted against a variation in tube voltage 114 where the anatomy being imaged may include vascular tissue filled with iodine-containing contrast agent. As previously noted with reference to FIG. 4, response curve 116 represents a variation of the optimal CNR² per skin-dose 112 as a function of the tube voltage 114 for an OEW, where w=Δμ, while response curve 118 embodies a variation of the optimal CNR² per skin-dose 112 as function of the tube voltage 114 for an OEW, where w=1/E³. Additionally, response curve 120 corresponds to a variation of the optimal CNR² per skin-dose 112 as a function of the tube voltage 114 for a photon counting system, where w=1, while response curve 122 signifies a variation of optimal CNR² per skin-dose 112 as a function of the tube voltage 114 for a conventional energy integrating system, where w=E. Furthermore, reference numeral 124 embodies an optimum region of operating the tube. In the embodiment depicted in FIG. 5, an optimum tube voltage for imaging vascular tissue may be in a range from about 80 kVp to about 120 kVp. Furthermore, OEW processing algorithms are optimum. As previously noted, this information regarding the optimum tube voltage may be added to the IQ optimization tables 78 (see FIG. 3). In addition, reference numeral 126 embodies a region of tube saturation. Accordingly, the system 10 (see FIG. 1) may be scanned at a relatively slow rate to facilitate the acquisition of a greater number of photons due to tube limitation.

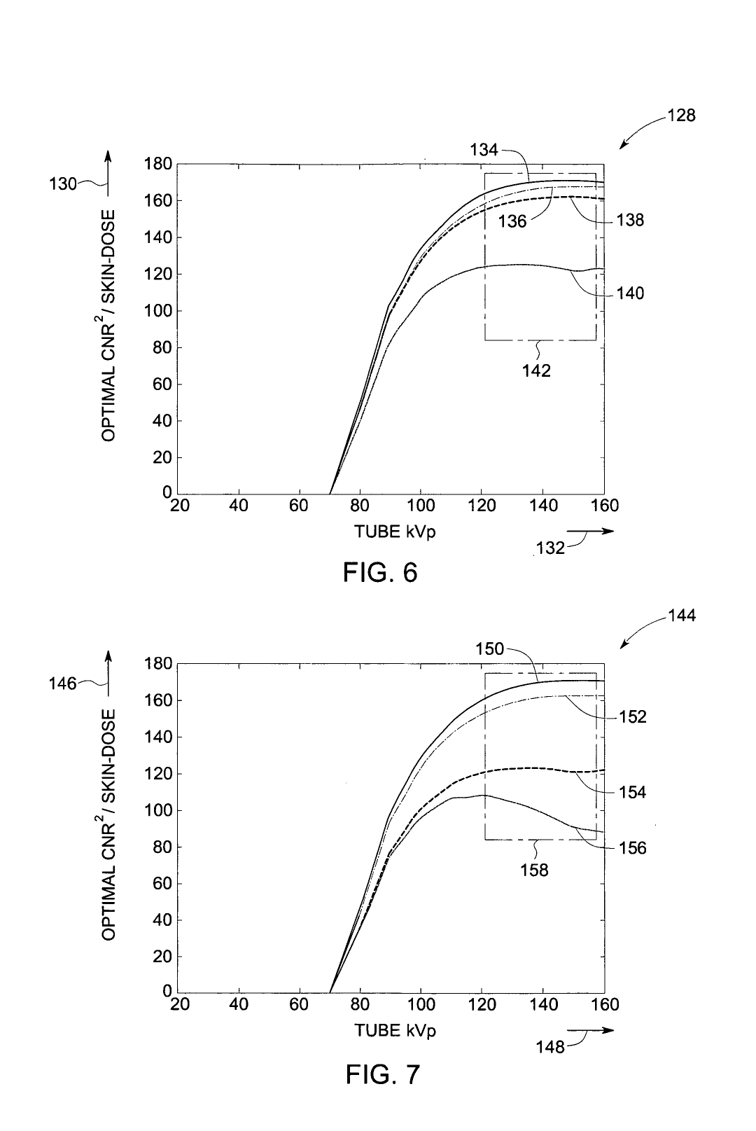

Turning now to FIG. 6, a graphical representation of simulation results 128 depicting a variation in an optimal CNR² per skin-dose 130 is plotted against a variation in tube voltage 132 where the anatomy being imaged is soft tissue such as muscle. As previously noted with reference to FIGS. 4-5, response curve 134 represents a variation of the optimal CNR² per skin-dose 130 as a function of the tube voltage 132 for an OEW, where w=Δμ, while response curve 136 embodies a variation of the optimal CNR² per skin-dose 130 as function of the tube voltage 132 for an OEW, where w=1/E³. Additionally, response curve 138 corresponds to a variation of the optimal CNR² per skin-dose 130 as a function of the tube voltage 132 for a photon counting system, where w=1, while response curve 140 signifies a variation of optimal CNR² per skin-dose 130 as a function of the tube voltage 132 for a conventional energy integrating system, where w=E. Reference numeral 142 embodies an optimal region of operating the tube. In the embodiment depicted in FIG. 6, an optimum tube voltage for imaging soft tissue may be in a range from about 120 kVp to about 160 kVp. It should be noted that the OEW algorithm associated with response curve 134 is an optimal bin processing algorithm. As previously noted, this information regarding the optimum tube voltage may be added to the IQ optimization tables 78 (see FIG. 3).

FIG. 7 illustrates a graphical representation 144 depicting a variation in an optimal CNR² per skin-dose 146 is plotted against a variation in tube voltage 148 where the anatomy being imaged is fat. As previously noted with reference to FIGS. 4-6, response curve 150 represents a variation of the optimal CNR² per skin-dose 146 as a function of the tube voltage 148 for a OEW, where w=Δμ=μ_{fat}-μ_{water}, while response curve 152 embodies a variation of the optimal CNR² per skin-dose 146 as function of the tube voltage 148 for a OEW, where w=1/E³. Additionally, response curve 154 corresponds to a variation of the optimal CNR² per skin-dose 146 as a function of the tube voltage 148 for a photon counting system, where w=1, while response curve 156 signifies a variation of optimal CNR² per skin-dose 146 as a function of the tube voltage 148 for a conventional energy integrating system, where w=E. Moreover, reference numeral 158 embodies an optimal region of operating the tube. In the embodiment depicted in FIG. 7, an optimum tube voltage for imaging fat may be in a range from about 120 kVp to about 160 kVp. Further, it should be noted that the OEW algorithm associated with response curve 150 is an optimal bin processing algorithm. As previously noted, this information regarding the optimum tube voltage may be added to the IQ optimization tables 78 (see FIG. 3).

As will be appreciated by those of ordinary skill in the art, the foregoing examples, demonstrations, and process steps may be implemented by suitable code on a processor-based system, such as a general-purpose or special-purpose computer. It should also be noted that different implementations of the present technique may perform some or all of the steps described herein in different orders or substantially concurrently, that is, in parallel. Furthermore, the functions may be implemented in a variety of programming languages, such as C++ or Java. Such code, as will be appreciated by those of ordinary skill in the art, may be stored or adapted for storage on one or more tangible, machine readable media, such as on memory chips, local or remote hard disks, optical disks (that is, CD's or DVD's), or other media, which may be accessed by a processor-based system to execute the stored code. Note that the tangible media may include paper or another suitable medium upon which the instructions are printed. For instance, the instructions may be electronically captured via optical scanning of the paper or other medium, then compiled, interpreted or otherwise processed in a suitable manner if necessary, and then stored in a computer memory.

The various methods for enhancing image quality and systems for enhancing image quality described hereinabove facilitate optimization of the acquisition protocol, reconstruction, processing and visualization algorithms to achieve enhanced CNR² per unit dose based on information related to patient size and shape, and intended anatomical targets, thereby advantageously preventing high patient dose. Consequently, the CNR is relatively higher for the same dose or same CNR may be achieved for a comparatively lower dose. Alternatively, the volume of contrast agent may be reduced by such methods. In addition, the methods described hereinabove facilitate control of flux rate to the detector by allowing for selection of a scan speed. For example, a relatively slower scan speed may be selected to ensure that the X-ray flux rate at the detector does not exceed some predetermined threshold and other preferred or optimal system acquisition settings and preferences. Furthermore, the acquisition protocols and image processing algorithms may be adapted to the intended target tissue of the user.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method of enhancing image quality and providing tissue composition information by analysis of energy discrimination data, the method comprising:
determining a radiation dosage at one or more energy spectrum levels based on patient parameters and user selected parameters.

2. The method of claim 1, wherein the determining step comprises:
acquiring patient parameters;
receiving user selected parameters;
selecting system acquisition settings based on the patient parameters and the user selected parameters;
acquiring image data from the patient based on the selected settings; and
processing the image data.

3. The method of claim 2, wherein the patient parameters are representative of estimates of patient size, shape, and anatomy being imaged.

4. The method of claim 2, wherein the user selected parameters are representative of image diagnostic exam type and visualization mode.

5. The method of claim 2, wherein the selecting step further comprises determining system acquisition settings from multi-dimensional look-up tables based on the patient parameters and the user selected parameters.

6. The method of claim 5, wherein the system acquisition settings comprise a desirable scan rate, a source filter material and thickness, a tube voltage, a current, or combinations thereof.

7. The method of claim 5, further comprising estimating incident flux for each view and each energy bin.

8. The method of claim 2, wherein the processing step comprises:
correcting the acquired image data;
weighting energy bin values to achieve higher contrast-to-noise or composition information;
reconstructing an image using corrected image data to generate a reconstructed image;
correcting image data using the reconstructed image;
applying post-processing algorithms to the reconstructed image to generate a final image; and
presenting the final image to user.

9. The method of claim 8, where the correcting the acquired image data step comprises:
acquiring image data from a first region of a pixel, wherein the first region has a first area;
acquiring image data from a second region of the pixel, wherein the second region has a second area; and
combining image data from the first and second regions to obtain composite image data for the pixel.

10. The method of claim 9, wherein each of the first and second regions is configured to count photons received and associate an energy bin to each photon counted.

11. The method of claim 9, further comprising calibrating the image data from each of the first and second regions to generate calibrated data.

12. The method of claim 11, wherein calibrating the image data comprises:
measuring detected charge as a function of input X-ray flux; and
fitting the detected charge to a polynomial function.

13. The method of claim 12, wherein the fitting step comprises correcting pile-up effects, non-ideal detector response characteristics or both in the image data acquired from each of the first and second regions.

14. The method of claim 9, wherein the combining step comprises combining the image data from the first and second regions weighted inversely by associated noise variance.

15. A system for enhancing image quality and providing tissue composition information by analysis of energy discrimination data, the system configured to determine a radiation dosage at one or more energy spectrum levels based on patient parameters and user selected parameters, wherein the system is configured to:
acquire patient parameters;
receive user selected parameters;
select system acquisition settings based on the patient parameters and the user selected parameters, by determining the system acquisition settings from multi-dimensional look-up tables based on the patient parameters and the user selected parameters, and wherein the system acquisition settings comprise a desirable scan rate, a source filter material and thickness, a tube voltage, a current or combinations thereof;
acquire image data from the patient based on the selected settings; and
process the image data.

16. A system for enhancing image quality and providing tissue composition information by analysis of energy discrimination data, the system configured to determine a radiation dosage at one or more energy spectrum levels based on patient parameters and user selected parameters.

17. The system of claim 16, wherein the system is configured to:
acquire patient parameters;
receive user selected parameters;
select system acquisition settings based on the patient parameters and the user selected parameters;
acquire image data from the patient based on the selected settings; and
process the image data.

18. The system of claim 17, wherein the patient parameters are representative of estimates of patient size, shape, and anatomy being imaged.

19. The system of claim 17, wherein the user selected parameters representative of image diagnostic exam type and visualization mode.

20. The system of claim 17, wherein the system acquisition settings comprise a desirable scan rate, a source filter material and thickness, a tube voltage, a current, or combinations thereof.

21. The system of claim 17, wherein the control system is further configured to determine system acquisition settings from multi-dimensional look-up tables based on the patient parameters and user selected parameters.

22. A computer readable medium comprising one or more tangible media, wherein the one or more tangible media comprise:
code adapted to determine a radiation dosage at one or more energy spectrum levels based on patient parameters and user selected parameters.

23. The computer readable medium, as recited in claim 22, wherein the code adapted to determine a radiation dosage comprises:
code adapted to acquire patient parameters;
code adapted to receive user selected parameters;
code adapted to select system acquisition settings based on the patient parameters and the user selected parameters;
code adapted to acquire image data from the patient based on the selected settings; and
code adapted to process the image data.

24. A radiographic imaging system comprising:
a radiation source configured to emit a stream of radiation toward a patient to be scanned;
a control system configured to enhance image quality and providing tissue composition information by analysis of energy discrimination data by determining a radiation dosage based on patient parameters and user selected parameters;
a detector assembly configured to detect the stream of radiation and to generate one or more signals responsive to the stream of radiation, wherein the detector assembly comprises one or more detectors configured to absorb radiation;
a system controller configured to rotate the radiation source and the detector assembly and to acquire one or more sets of projection data from the one or more detectors via a data acquisition system; and
a computer system operationally coupled to the radiation source and the detector assembly, wherein the computer system is configured to receive the one or more sets of projection data.

25. The system of claim 24, wherein the control system is configured to:
acquire patient parameters;
receive user selected parameters;
select system acquisition settings based on the patient parameters and the user selected parameters;
acquire image data from the patient based on the selected settings; and
process the image data.
